(19) United States
(12) Patent Application Publication
(10) Pub. No.: US 2018/0021104 A1
(43) Pub. Date: Jan. 25, 2018

(54) DENTAL LASER SYSTEM AND METHOD

(71) Applicant: Biolase, Inc., Irvine, CA (US)

(72) Inventors: MATT DUNCAN, SAN CLEMENTE, CA (US); DMITRI BOUTOUSSOV, Dana Point, CA (US)

(73) Assignee: Biolase, Inc., IRVINE, CA (US)

(21) Appl. No.: 15/655,164

(22) Filed: Jul. 20, 2017

Related U.S. Application Data
(60) Provisional application No. 62/364,782, filed on Jul. 20, 2016.

(51) Int. Cl. A61C 1/00; G06F 3/0484; A61N 5/06

(52) U.S. Cl. CPC A61C 1/0046; A61N 5/0601; A61C 1/0007; G06F 3/0484; A61B 18/20

(57) ABSTRACT

Some embodiments of the invention include a dental laser system including a mobile transportation cart with a cabinet and drawers, and wheels. An adjustment column is coupled to at least one height adjustable table that is configured and arranged to be raised and lowered. In some embodiments, the dental laser assembly is removably supported or mounted on the table, and includes a dental laser, a laser tool or handpiece coupled to the dental laser, and a display assembly mounted in a display mount of the dental laser. In some embodiments, the distance between the dental laser assembly and the cabinet is dependent on an adjusted position of the table. Further, in some embodiments, the display assembly is configured to be pivoted, rotated or tilted, and the display mount includes at least one retractable or moveable grip.

### RELATED APPLICATIONS

This application claims the benefit, and priority benefit, of U.S. Provisional Patent Application Ser. No. 62/364,782, filed Jul. 20, 2016, the disclosure and contents of which are incorporated by reference herein in their entirety.

### BACKGROUND

The increasing range and sophistication of dental tools has broadened their appeal within the dental community. Many dental practices usually have at least one complex dental laser station that often includes control systems, displays, and one or more user interfaces of various forms that can be used to control the tool, and/or monitor and communicate some function or operational characteristic of the tool.

In most systems available on the market today, the dental tool is not configured for optimization of the user experience. For example, if the dental laser tool includes a controller, the display interface is typically not configured for ease of positioning. Similarly, any laser tool may not be integrated with or coupled to the dental tool for ease of use and storage. Furthermore, the dental tool may not be integrated with a station that can house clinical supplies and enable the dental tool to be transported and positioned for clinical practice. Moreover, the dental laser tool is often not easily maneuvered or positioned ergonomically with respect to the user.

### SUMMARY

Some embodiments include a dental laser system comprising a mobile transportation cart including cabinet or drawer assembly and at least one support table, an adjustment column coupled to the cabinet or drawer assembly, and at least one support table coupled to or mounted on the adjustment column. In some embodiments, the at least one support table is configured and arranged to be raised and lowered with respect cabinet or drawer assembly using the adjustment column. Some embodiments include a dental laser assembly removably supported or mounted on the support table. In some embodiments, the dental laser assembly includes at least one dental laser, at least one laser tool or handpiece coupled to the at least one dental laser, and at least one display assembly coupled to the at least on dental laser. In some embodiments, the distance between the dental laser assembly and the cabinet or drawer assembly is dependent on an adjusted position of the at least one support table.

In some embodiments, the cabinet or drawer assembly includes at least one drawer. In some further embodiments, the cabinet or drawer assembly includes at least one storage cabinet. In some embodiments, the cabinet or drawer assembly includes at least one storage cabinet door. In some embodiments, the mobile transportation cart comprises a set of wheels, that can enable rolling movement of the cart by a user.

In some embodiments, the support table includes at least one transportation handle. In some embodiments, the least one transportation handle extends from the support table and forms an aperture. In some embodiments, the at least one transportation handle extends from the support table generally perpendicular to the adjustment column. In some embodiments, the at least one transportation handle extends from one side of the support table to the opposite side of the support table. In some embodiments, the at least one transportation handle extends from one side of the dental laser assembly to the opposite side of the dental laser assembly.

In some embodiments of the invention, the at least one display assembly comprises a display of a computing device or controller. In some embodiments, the computing device or controller comprises a mobile device that is attachable and detachable from the dental laser assembly. In some embodiments, the mobile device comprises at least one of a laptop computer, tablet computer, or smart phone.

Some embodiments include a laser tool mount configured to mount the laser tool or handpiece to the display assembly. Some embodiments include a display mount configured to enable mounting of the display assembly. In some embodiments, the display mount comprises at least one fixed or moveable lower grip. In some embodiments, the display mount includes at least one retractable upper grip. In some embodiments, the display assembly is configured to be tilted, pivoted or rotated on the dental laser assembly.

Some embodiments of the invention include a dental laser system comprising a mobile transportation cart including a cabinet and a plurality of drawers, a set of wheels enabling rolling movement of the cart by a user, and an adjustment column coupled to at least one height adjustable table, the at least one height adjustable table configured and arranged to be raised and lowered using the adjustment column. In some embodiments, the dental laser assembly is removably supported or mounted on the at least one height adjustable table, and includes at least one dental laser, at least one laser tool or handpiece coupled to the at least one dental laser, and at least one display assembly mounted in a display mount of the at least on dental laser. In some embodiments, the distance between the dental laser assembly and the cabinet is dependent on an adjusted position of the at least one height adjustable table. Further, in some embodiments, the display assembly is configured to be pivoted, rotated or tilted, and the display mount includes at least one retractable or moveable grip.

### DESCRIPTION OF THE DRAWINGS

FIG. 1A illustrates a perspective view of a dental laser station with a dental laser device positioned in a lowered state in accordance with some embodiments of the invention.

FIG. 1B illustrates a perspective view of a dental laser station with a dental laser device positioned in an upper or raised state in accordance with some embodiments of the invention.

FIG. 2A illustrates a side view of a dental laser device including a display assembly in a lowered position in accordance with some embodiments of the invention.

FIG. 2B illustrates a side view of a dental laser device including a display assembly in a raised position in accordance with some embodiments of the invention.

FIG. 3 illustrates a rear perspective view of a display assembly in accordance with some embodiments of the invention.

FIG. 4 illustrates a front perspective view of a dental laser device in accordance with some embodiments of the invention.

FIG. 5 illustrates a front-side perspective view of the dental laser station of FIG. 1A in accordance with some embodiments of the invention.

FIG. 6 illustrates a side perspective view of the dental laser station of FIG. 1A in accordance with some embodiments of the invention.

FIG. 7 illustrates a front-side perspective view of the dental laser station of FIG. 1A with a laser fiber rotated to a use position in accordance with some embodiments of the invention.

FIG. 8 illustrates a front-side perspective view of the dental laser station of FIG. 1A with a laser fiber rotated to a store or transport position in accordance with some embodiments of the invention.

FIG. 9 illustrates a perspective view of a dental laser station in accordance with some further embodiments of the invention.

FIG. 10 illustrates a perspective view of a dental laser station in accordance with some further embodiments of the invention.

FIG. 11 illustrates a side view of a dental laser station in accordance with some further embodiments of the invention.

FIG. 12 illustrates a side view of a dental laser station in accordance with some further embodiments of the invention.

FIG. 13 illustrates a front view of a dental laser station in accordance with some further embodiments of the invention.

FIG. 14 illustrates a rear view of a dental laser station in accordance with some further embodiments of the invention.

FIG. 15 illustrates a top view of a dental laser station in accordance with some further embodiments of the invention.

FIG. 16 illustrates a bottom view of a dental laser station in accordance with some further embodiments of the invention.

### DETAILED DESCRIPTION

Before any embodiments of the invention are explained in detail, it is to be understood that the invention is not limited in its application to the details of construction and the arrangement of components set forth in the following description or illustrated in the following drawings. The invention is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Unless specified or limited otherwise, the terms “mounted,” “connected,” “supported,” and “coupled” and variations thereof are used broadly and encompass both direct and indirect mountings, connections, supports, and couplings. Further, “connected” and “coupled” are not restricted to physical or mechanical connections or couplings.

The following discussion is presented to enable a person skilled in the art to make and use embodiments of the invention. Various modifications to the illustrated embodiments will be readily apparent to those skilled in the art, and the generic principles herein can be applied to other embodiments and applications without departing from embodiments of the invention. Thus, embodiments of the invention are not intended to be limited to embodiments shown, but are to be accorded the widest scope consistent with the principles and features disclosed herein. The following detailed description is to be read with reference to the figures, in which like elements in different figures have like reference numerals. The figures, which are not necessarily to scale, depict selected embodiments and are not intended to limit the scope of embodiments of the invention. Skilled artisans will recognize the examples provided herein have many useful alternatives that fall within the scope of embodiments of the invention.

The various embodiments of the invention described herein include devices, apparatus, systems and methods to enable the use and control and/or monitoring of complex dental laser procedures. Further, embodiments of the invention include control systems, displays, and associated user interfaces that can be used to control some aspect of a dental laser station, and/or communicate some function or operational characteristic of the tool to the user. For example, some embodiments can include control systems, displays, and user interfaces that can be used to perform procedures related to dental restoration and oral disease prevention. More specifically, some embodiments of the invention described herein include devices, apparatus, systems and methods to enable a user to operate and/or monitor the functional aspects of dental laser stations using interfaces that can be customized for a specific dental procedure and/or for any specific dental specialty including, but not limited to general dentistry, oral and maxillofacial dentistry, orthodontic dentistry, endodontic dentistry, pediatric dentistry, cosmetic dentistry, and so on.

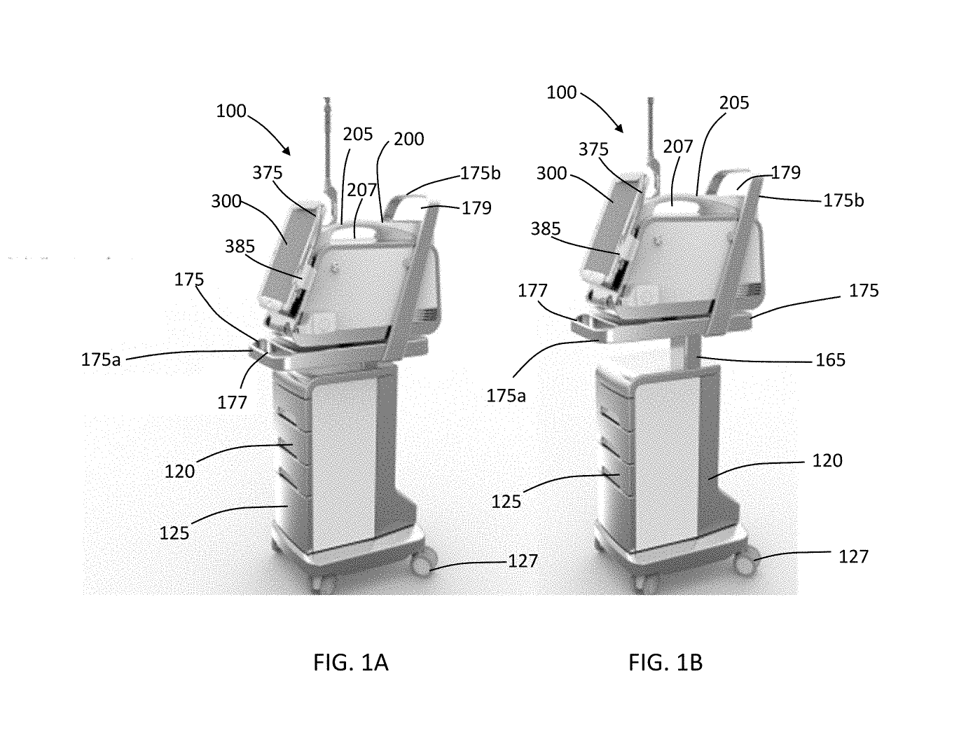

Some embodiments include dental lasers and/or dental laser stations that can include mechanisms for positioning a dental laser or dental station (e.g., such as for moving a dental laser or dental laser station up or down, side, to side, etc., in order to position the equipment as necessary or required by the user. For example, FIG. 1A illustrates a perspective view of a dental laser station 100 with a dental laser device 200 positioned in a lowered state in accordance with some embodiments of the invention. Some further embodiments of the invention include dental lasers and/or dental laser stations that are mounted to moveable tables, mounts or carts. For example, in some embodiments, the dental laser station 100 can include a cart 120, and/or a dental laser device 200 can be mounted or supported on the cart 120. In some embodiments, the cart 120 can include a table 175 or other conventional support structure capable of supporting one or more dental laser devices 200 and/or one or more additional components, devices, or supplies. In some embodiments of the invention, the cart 120 can include one or more conventional batteries or other power supply modules. In some embodiments, any mounted or coupled conventional batteries or other power supply modules can provide a sustained, intermittent or on-demand power for the dental laser device 200, or any device or component coupled to or associated with the dental laser device 200. In some other embodiments, the cart 120 can include at least one conventional compressor and/or at least one source of conventional compressed gas. In some other embodiments, the cart 120 can include at least one conventional water cleaning and sterilization module. Further, in some embodiments, the cart 120 can include a source of sterile water or other conventional dental aspiration fluid and/or at least one disinfecting fluid, or combinations thereof.

In some embodiments of the invention, the cart 120 can comprise a cabinet and/or drawer assembly 125 including drawers and/or cabinet space for storage of miscellaneous supplies, tools, and/or accessories. In some embodiments, using a vertical adjustment, the height of the cart 120 can be adjusted to enable the table 175 to be raised and lowered with respect the cabinet or drawer assembly 125. For example, FIG. 1B illustrates a perspective view of a dental laser station 100 with a dental laser device 200 positioned in an upper or raised state in accordance with some embodiments of the invention. Some embodiments include an adjustment column 165 that can be used to facilitate changing the distance of the table 175 with respect to the top end of the cabinet or drawer assembly 125. In some embodiments, the table 175 can be fixed to the adjustment column 165, and the column 165 can be configured to slide into and out of the cabinet or drawer assembly 125. In some other embodiments, the column 165 can be configured in a fixed position with respect to the assembly 125, and the table 175 can be raised and lowered relative to the column 165 with respect to the assembly 125. In some embodiments, the length of the column 165 or the travel distance of the table 175 can be extended or reduced to enable the distance between the table 175 and the assembly 125 to vary depending on a users's requirements. In other words, the distance between the table 175 and the assembly 125 on the cart 120 shown in the non-limiting embodiment of FIG. 1B can be varied so that the distance is smaller or larger than illustrated.

In some embodiments of the invention, the dental laser station 100 can include a dental laser device 200 that includes a display assembly 300. In some embodiments of the invention, the display assembly 300 can include a graphical user interface (hereafter “GUI”) comprising one or more displayed menus that can be navigated by user to control or monitor one or more functions of a dental laser. Displayed menus can include category buttons, any one of which can have one or more control system attributes. These category buttons may be defined as, but not limited to, dentin, enamel, anterior deciduous, hemostasis, periodontic, endodontic, incision/excision, de-sensitization and osseous. In some embodiments, the display assembly 300 can comprise “soft” buttons that can be graphically rendered in a GUI, and/or can be hard buttons adjacent to the displays on a dental tool or associated control equipment, and/or a remote control or a WiFi linked system. In some embodiments, the display assembly 300 comprises a touchscreen display. For example, in some embodiments, the display can comprise a touchscreen display configured to enable a user to interact with the displayed GUI. In some embodiments, user interactions with the GUI can include contact of at least a portion of the display to initiate or represent an input to the display and/or an input or selection of any information within the display or GUI. For example, in some embodiments, a user, using single, multiple, or repeated physical contact with the display can initiate one or more functions of the dental laser station. In some embodiments, using one or more portions of the GUI, a user can enter, select, and/or modify one or more system or operational variables or attributes. For example, using at least one displayed feature, a user can use the GUI to control a plurality of system or operational variables or attributes. These parameters can be modified interactively to adjust and optimize the operational characteristics of a dental laser prior to starting a dental procedure, during a dental procedure, and/or after a dental procedure has been performed.

In some embodiments, the dental laser of the dental laser device 200 of the dental laser station 100 can include a variety of different lasers, laser diodes, or other sources of light. In some embodiments, the laser sources can include an erbium, yttrium, aluminum garnet (Er:YAG) solid state laser; a chromium, thulium, erbium, yttrium, aluminum garnet (CTE:YAG) solid state laser; an erbium, yttrium orthoaluminate (Er:YAL0₃) solid state laser; a holmium, yttrium, aluminum garnet (Ho:YAG) solid state laser; a quadrupled neodymium, yttrium, aluminum garnet (quadrupled Nd:YAG) solid state laser; an excimer laser; or a carbon dioxide (CO₂) laser. In some further embodiments of the invention, the dental laser can include one or more erbium, chromium, yttrium, scandium, gallium garnet lasers (Er:Cr:YSGG).

In some embodiments, the dental laser station 100 can include a removable or replaceable conventional laptop or tablet computer or other mobile computing device (e.g., such as a smart phone). In some embodiments, the cart 120 can include a mounting point for a conventional laptop or tablet computer or other mobile computing device that can be coupled to the cart 120 and/or mounted to the table 175. In some embodiments, the display assembly 300 can comprise a tablet display. In some embodiments, the conventional laptop or tablet computer or other mobile computing device can be used as a training and/or educational tool (e.g., for use as a display for educational videos and/or training materials including text, images, and/or video). Any of the educational videos and/or training materials can include accompanying sound that can be played through the tablet or other audio-capable component coupled to the station 100. In some further embodiments, the conventional laptop or tablet computer or other mobile computing device can be used as a communication tool. In some embodiments, a user can communicate to and/or from another user, a trainer, a doctor or dentist, and/or one or more patients using the conventional laptop or tablet computer or other mobile computing device.

In some embodiments of the invention, the vertical adjustment capability of the table 175 of the cart 120 can enable a user to achieve an optimal viewing angle of the display assembly 300 when seated or standing, or based on the viewing or access height of the user. For example, a user needing viewing or access at a lower height (represented in the example embodiment of FIG. 1A), or a user needing viewing or access at a greater height (e.g., represented in the example embodiment of FIG. 1B). For example, in some embodiments, the display assembly 300 can be raised by raising the table 175, thereby moving the dental laser device 200 including the display assembly 300 away from the assembly 125 (i.e., moving from the status of the dental laser station 100 shown in FIG. 1A to the status of the dental laser station 100 shown in FIG. 1B). In some embodiments, movement of the table 175 can be facilitated using the handle 175a or other conventional attachment, bar, or lever, etc. In some embodiments, the handle 175a can extend from the table 165 generally perpendicular to the adjustment column 165. In some embodiments, the user can grasp the handle 175a to raise or lower the table 175. In some further embodiments, the handle 175b can be used to facilitate raising or lowering of the table 175.

In some embodiments, the handle 175a can extend outwardly from the table 175 to facilitate a user handling the handle 175a. In some embodiments, the handle 175a can extend at least a partial width of the table 175. In some embodiments, the handle 175a can extend substantially the entire width of the table 175. For example, in some embodiments, one end of the handle 175a can extend from the table 175 on one side, and another end of the handle 175a can extend from the table 175 on the opposite side. In some embodiments of the invention, the handle 175a can extend from the table 175 forming an aperture or cavity 177. In some embodiments, the aperture or cavity 177 can be used by a user to grip the handle 175a.

In some embodiments, the handle 175b can extend outwardly from the table 175 to facilitate a user handling the handle 175b. In some embodiments, the handle 175b can extend at least a partial width of the table 175. In some embodiments, the handle 175b can extend substantially the entire width of the table 175. For example, in some embodiments, one end of the handle 175b can extend from the table 175 on one side, and another end of the handle 175b can extend from the table 175 on the opposite side. In some embodiments of the invention, the handle 175b can extend from the table 175 forming an aperture or cavity 179. In some embodiments, the aperture or cavity 179 can be used by a user to grip the handle 175b.

In some embodiments, the cart 120 can include wheel assemblies 127 or other conventional movement assemblies to enable the dental laser station 100 to be moved from one location to another location. In some embodiments of the invention, one or both of the handles 175a, 175b can be used to maneuver the dental laser station 100 by moving on the wheel assemblies 127. For example, in some embodiments, the handle 175a can be used to position the dental laser station 100. In some further embodiments, the handle 175b can be used to position the dental laser station 100. In some embodiments, the wheel assemblies 127 can be locked to prevent unintended movement of the dental laser station 100. In some embodiments, the wheel assemblies 127 can be temporarily or permanently removed to enable the dental laser station 100 to remain stationary.

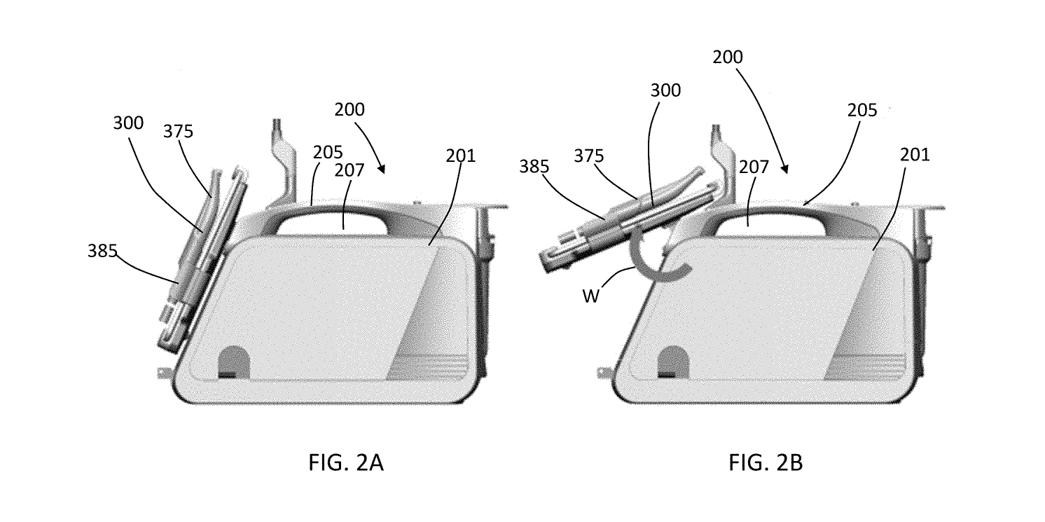

FIG. 2A illustrates a side view of a dental laser device 200 including a display assembly 300 in a lowered position in accordance with some embodiments of the invention. In some embodiments, the dental laser device 200 can include a display assembly 300 that can be titled, pivoted or rotated by a user to enable a mounted or coupled display device to be raised or lowered for optimal viewing angle. For example, FIG. 2B illustrates a side view of a dental laser device 200 including a display assembly 300 in a raised position, with arrow W representing a rotation arc of the display assembly 300 to the raised position from the lowered position shown in FIG. 2A. In some embodiments, the display assembly 300 can be tilted, pivoted or rotated to a greater or lesser angle than that depicted in FIG. 2B (e.g., to any intermediate positions between the lowered position shown in FIG. 2A, and the raised position shown in FIG. 2A). In some embodiments, the user can lower the assembly 300 so that the display device is positioned adjacent, proximate or flush with the main housing 201 of the dental laser device 200.

In some embodiments, the dental laser device 200 can include a transportation handle 205. In some embodiments, the transportation handle 205 can extend across the top end of the dental laser device 200. In some embodiments, the handle 205 can extend across the top end of the device 200 from the front end to the back end of the main housing 201 of the device 200 as shown. In some embodiments, the handle 205 can extend across the top end of the device 200 from the front end to the back end of the main housing 201 forming an aperture 207. In some embodiments, the display assembly 300 can be coupled to a front portion of the main housing 201 of the transportation handle 205. In some embodiments, the display assembly 300 can be coupled to the front portion of the housing of the transportation handle using a pivot or other conventional mechanism to enable the display assembly 300 to rotate or pivot.

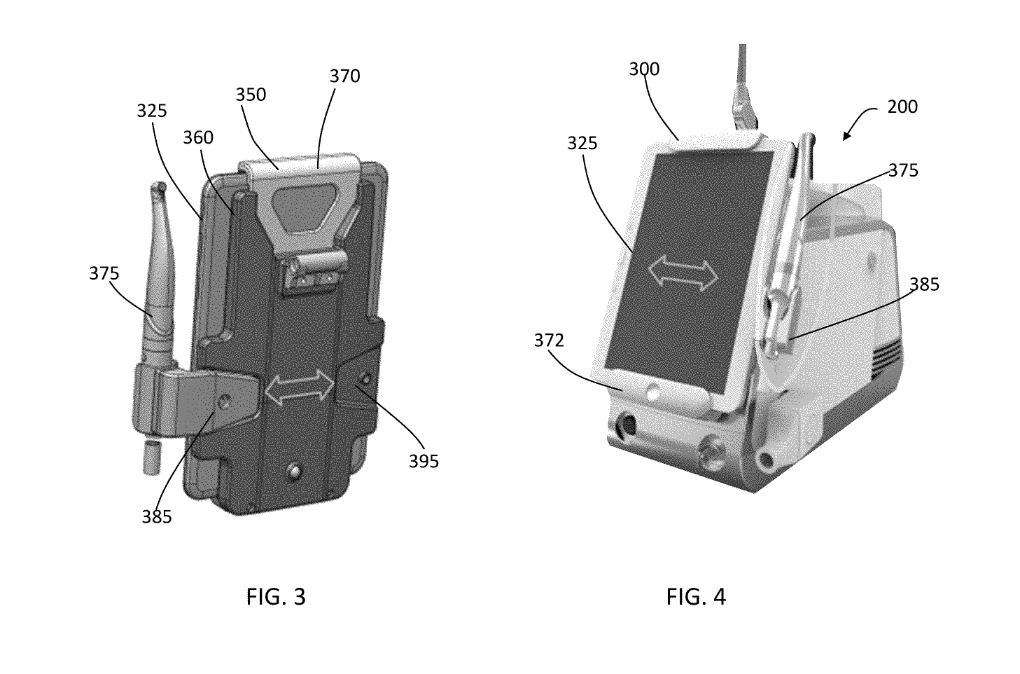

FIG. 3 illustrates a rear perspective view of a display assembly 300 in accordance with some embodiments of the invention. In some embodiments, the display assembly 300 can include a display mount assembly 350 including a display mount 360 and a coupled retractable grip 370. In some embodiments, a display device 325 can be mounted into the display mount 360 of the display mount assembly 350. In some further embodiments, the coupled retractable grip 370 can be used to grip and release the display device 325. In some embodiments, one or more laser tools can be mounted to the display assembly 300. For example, as shown in FIGS. 2A-2B, and FIGS. 3-4, some embodiments include a laser tool mount 385 configured to mount the laser tool or handpiece 375 to the display assembly 300. In some embodiments, the display assembly 300 can include mounting slots 395 configured to enable the laser tool mount 385 to be mounted to one or more sides of the display mount 360.

FIG. 4 illustrates a front perspective view of a dental laser device 200 in accordance with some embodiments of the invention. In some embodiments, the display device 325 can be mounted in the display mount 360 to enable the mounting position of the display device 325 to be adjusted by the user. In some embodiments, fixed or moveable lower grip 372 can be used to secure the display device 325 in the display mount 360. In some embodiments, the display mount 360 can enable the user to slide the display device 325 within the display mount 360 (e.g., where the display device 325 can be moved to left or right within the display mount 360).

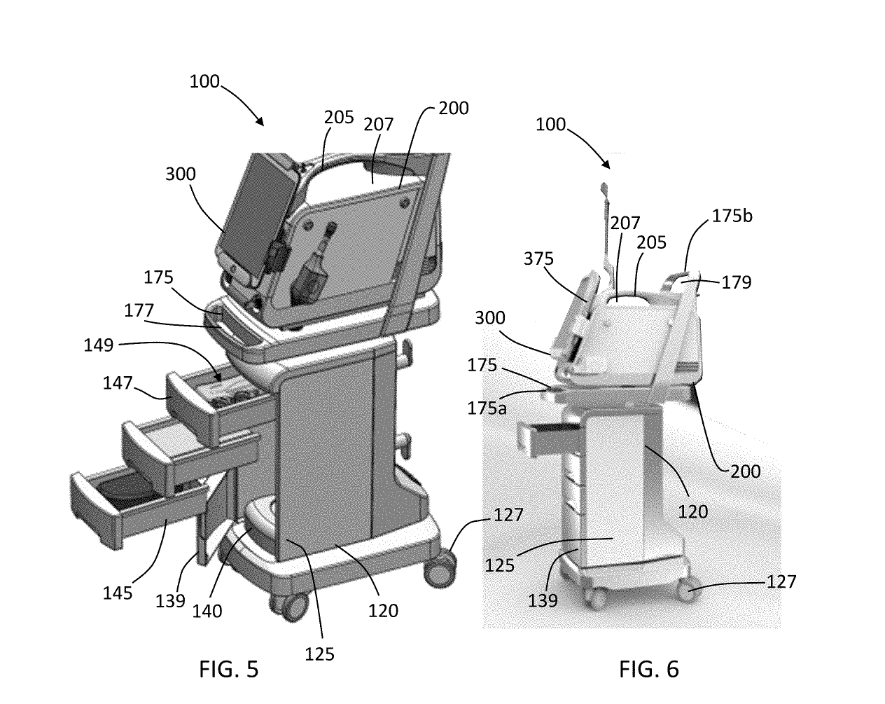

FIG. 5 illustrates a front-side perspective view of the dental laser station 100 of FIG. 1A in accordance with some embodiments of the invention. In some embodiments, the cart 120 can include a cabinet or drawer assembly 125 including a plurality of drawers 145 and cabinet space 140 for storage accessible using door 139. Some embodiments include three drawers and a single cabinet forming the space 140 and accessed using door 139. Some other embodiments can include additional or lesser numbers of drawers and/or partitions or other cabinets and spaces that those illustrated in the non-limiting embodiment of FIG. 5. FIG. 6 illustrates a side perspective view of the dental laser station 100 of FIG. 1A in accordance with some embodiments of the invention and shows a single open drawer of the plurality of drawers 145. In some embodiments, one or more drawers of the plurality of drawers 145 can be used to store supplies, tools, or accessories to assist in clinical practice using the dental laser station 100. For example, some embodiments include one or more laser handpiece tips, handpiece kit(s), glass, and/or a footswitch compartment (shown positioned as items 149 in top drawer 147).

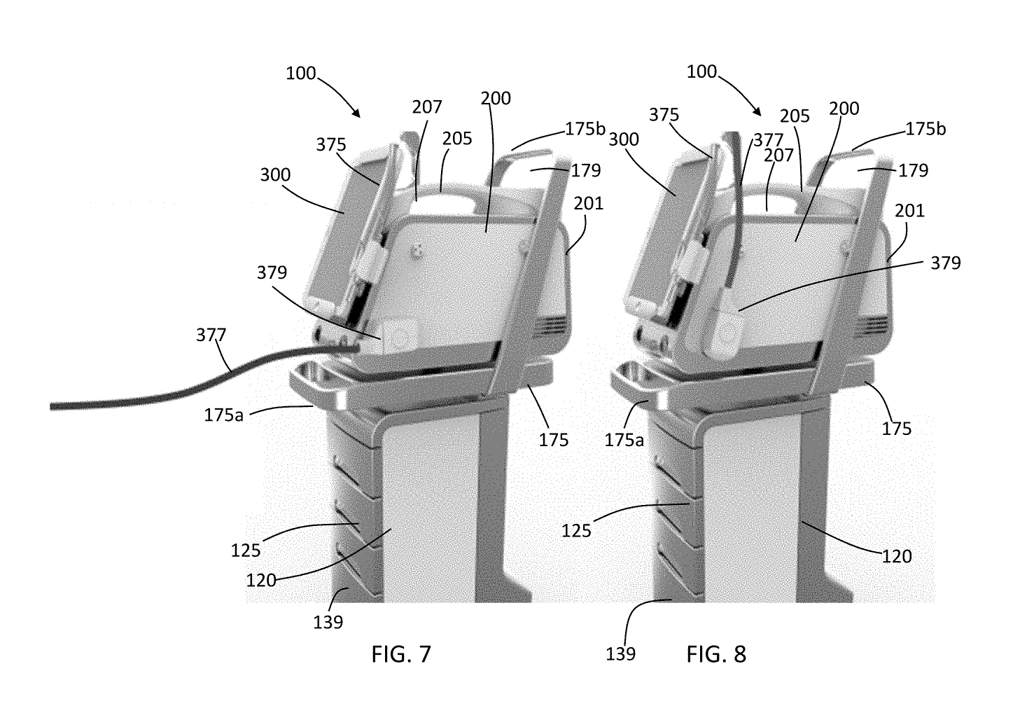

In some embodiments, the laser tool or handpiece 375 can comprise an articulating laser module that can enable a user to position a laser fiber for maximum range, comfort and balanced manipulation of the laser tool or handpiece 375 (e.g., the handpiece). For example, FIGS. 7 and 8 illustrate front-side perspective views of the dental laser station 100 of FIG. 1A in accordance with some embodiments of the invention. FIG. 7 illustrates a front-side perspective view of the dental laser station of FIG. 1A with a laser fiber rotated to a use position in accordance with some embodiments of the invention. The laser fiber 377 is shown extending away from the dental laser device 200 using a rotating or pivot coupling 379 in FIG. 7 in a use position, and is shown in a stored position in FIG. 8, with the laser fiber 377 adjacent or proximate the main housing 201 of the dental laser device 200. The laser fiber 377 can be retracted and stored for futher use at any time.

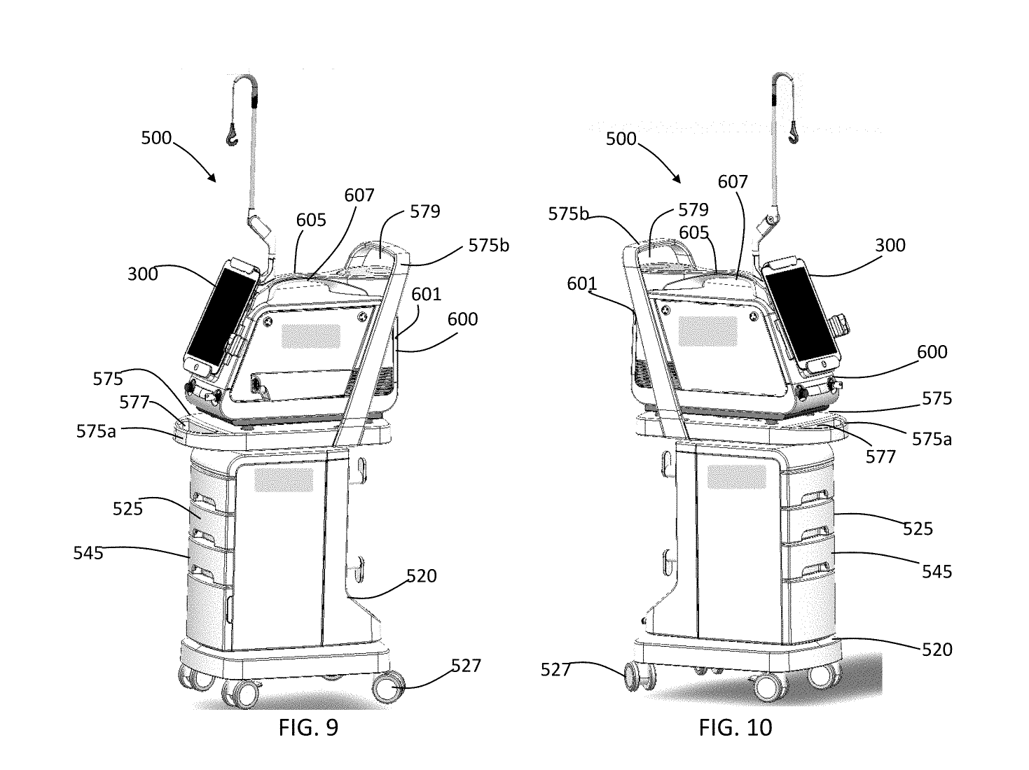

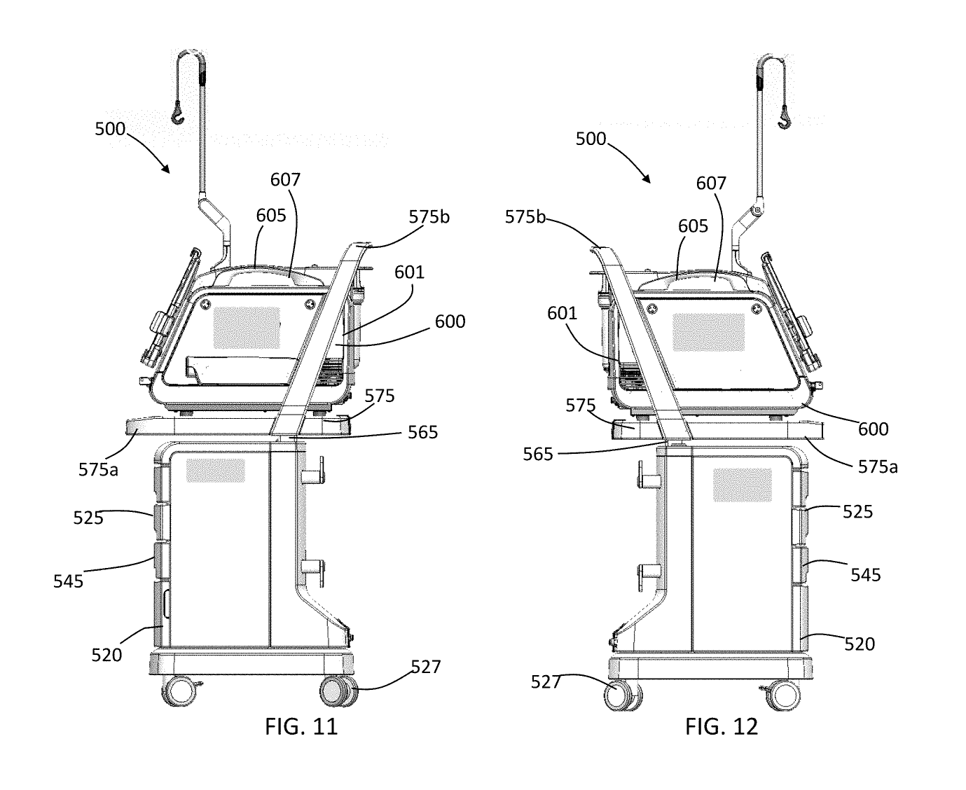

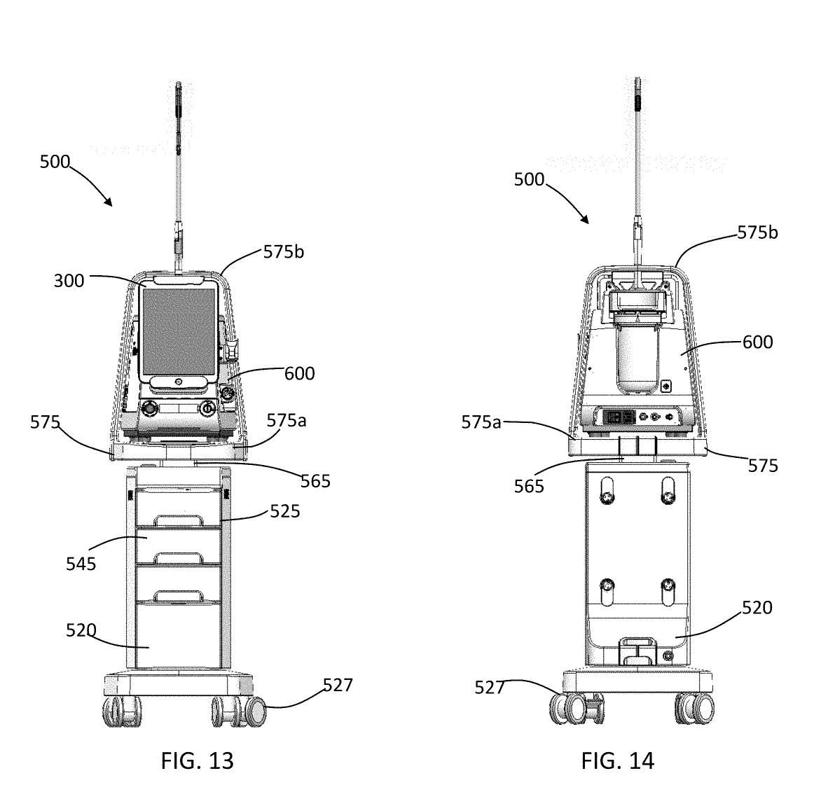

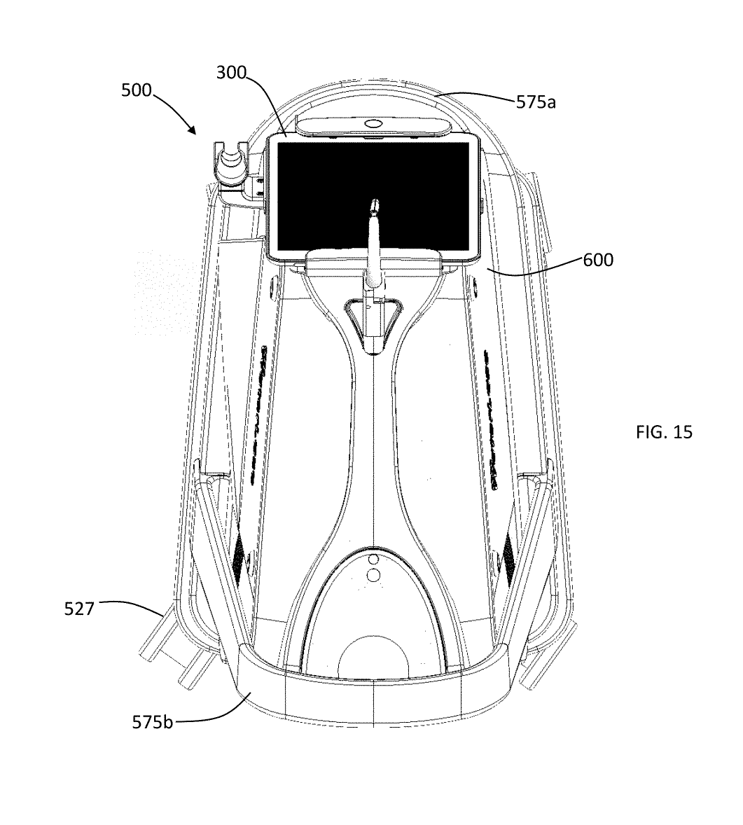

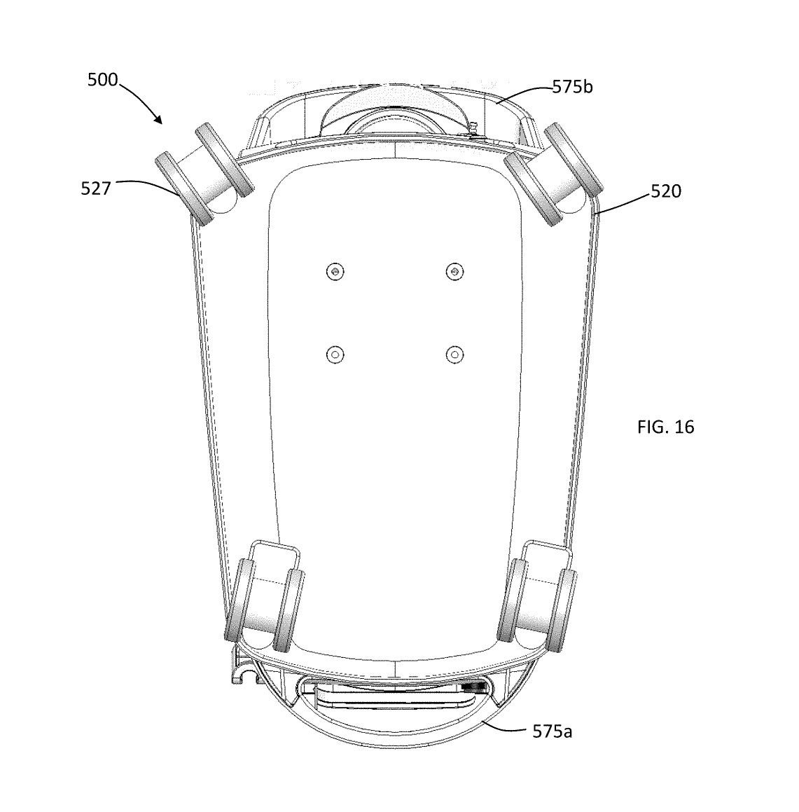

FIGS. 9 and 10 illustrate a perspective view of a dental laser station 500 in accordance with some further embodiments of the invention. FIGS. 11-12 illustrate side views of the dental laser station 500 in accordance with some further embodiments of the invention. Further, FIG. 13 illustrates a front view of a dental laser station 500 in accordance with some further embodiments of the invention, and FIG. 14 illustrates a rear view of a dental laser station 500 in accordance with some further embodiments of the invention. FIG. 15 illustrates a top view of a dental laser station 500 in accordance with some further embodiments of the invention. FIG. 16 illustrates a bottom view of a dental laser station 500 in accordance with some further embodiments of the invention.

FIG. 9 illustrates a perspective view of a dental laser station 500 in accordance with some further embodiments of the invention. In some embodiments, the dental laser station 500 can include a cart 520. Some embodiments include a dental laser device 600 mounted or supported on the cart 520. In some embodiments, the dental laser device 600 can include a main housing 601.

In some embodiments, the cart 520 can include a table 575 or other conventional support structure capable of supporting one or more dental laser devices 600 and/or one or more additional components, devices, or supplies. In some embodiments, the cart 520 can include at least one conventional battery or other power supply module. In some embodiments, the conventional battery or other power supply module can provide a sustained, intermittent or on-demand power for the dental laser device 600, or any other coupled or associated device or component. In some other embodiments, the cart 520 can include at least one conventional compressor and/or at least one source of conventional compressed gas. In some other embodiments, the cart 520 can include at least one conventional water cleaning and sterilization module. Further in some embodiments, the cart 520 can include a source of sterile water or other conventional dental aspiration fluid and/or at least one disinfecting fluid.

In some embodiments, the cart 520 can comprise a cabinet or drawer assembly 525 including drawers and cabinet space for storage of miscellaneous supplies, tools, and/or accessories. In some embodiments, using a vertical adjustment, the height of the cart 520 can be adjusted to enable the table 575 to be raised and lowered with respect the cabinet or drawer assembly 525. For example, in some embodiments, the dental laser station 500 with a dental laser device 600 can be positioned in an upper or raised state in accordance with some embodiments of the invention. To accomplish this, some embodiments include an adjustment column 565 that can be used to facilitate changing the distance of the table 575 with respect to the top end of the cabinet or drawer assembly 525 (e.g., see FIGS. 11-12). In some embodiments, the table 575 can be fixed to the adjustment column 565, and the adjustment column 565 can be configured to slide into and out of the cabinet or drawer assembly 525. In other embodiments, the adjustment column 565 can be configured in a fixed position with respect to the assembly 525, and the table 575 can be raised and lowered relative to the adjustment column 565 with respect to the assembly 525.

In some embodiments of the invention, the dental laser station 500 can include a dental laser device 600 that includes the display assembly 300. In some embodiments, the display can include at least some or all of the features and operational characteristics as described above with respect to the dental laser station 100. For example, in some embodiments of the invention, the display assembly 300 can include a graphical user interface (hereafter “GUI”) comprising one or more displayed menus that can be navigated by user to control or monitor one or more functions of a dental laser. In some embodiments, the laser sources of the dental laser device 600 of the station 500 can include an erbium, yttrium, aluminum garnet (Er:YAG) solid state laser; a chromium, thulium, erbium, yttrium, aluminum garnet (CTE:YAG) solid state laser; an erbium, yttrium orthoaluminate (Er:YAL03) solid state laser; a holmium, yttrium, aluminum garnet (Ho:YAG) solid state laser; a quadrupled neodymium, yttrium, aluminum garnet (quadrupled Nd:YAG) solid state laser; an excimer laser; or a carbon dioxide (CO2) laser. In some further embodiments of the invention, the dental laser can include one or more erbium, chromium, yttrium, scandium, gallium garnet lasers (Er:Cr:YSGG).

In some further embodiments, the dental laser station 500 can include a removable conventional laptop or tablet computer or other mobile computing device (e.g., such as a smart phone). For example, in some embodiments, the cart 520 can include a mounting point for a conventional tablet or other mobile computing device that can be coupled to the cart 520 and/or mounted to the table 575. In some embodiments, the display assembly 300 can comprise a tablet display. In some embodiments, the tablet or other mobile computing device can be used as a training and/or educational tool (e.g., for use as a display for educational videos and/or training materials including text, images, and/or video). Any of the educational videos and/or training materials can include accompanying sound that can be played through the tablet or other audio-capable component coupled to the station 500. In some further embodiments, the tablet or other mobile computing device can be used as a communication tool. In some embodiments, a user can communicate to and/or from another user, a trainer, a doctor or dentist, and/or one or more patients using the tablet or other mobile computing device.

In some embodiments, the vertical adjustment capability of the table 575 of the cart 520 can enable a user to achieve an optimal viewing angle of the display assembly 300 when seated, or when standing. For example, in some embodiments, the display assembly 300 can be raised by raising the table 575, thereby moving the dental laser device 600 including the display assembly 300 away from the assembly 525. In some embodiments, movement of the table 575 can be facilitated using the positioning handle 575a or other conventional attachment, bar, or lever, etc. For example, in some embodiments, the user can grasp the positioning handle 575a to raise or lower the table 575. In some further embodiments, the transportation handle 575b can also be used to facilitate raising or lowering of the table 575.

In some embodiments, the handle 575a can extend outwardly from the table 575 to facilitate a user handling the handle 575a. In some embodiments, the handle 575a can extend at least a partial width of the table 575. In some embodiments, the handle 575a can extend substantially the entire width of the table 575. For example, in some embodiments, one end of the handle 575a can extend from the table 575 on one side, and another end of the handle 575a can extend from the table 575 on the opposite side. In some embodiments of the invention, the handle 575a can extend from the table 575 forming an aperture or cavity 577. In some embodiments, the aperture or cavity 577 can be used by a user to grip the handle 575a.

In some embodiments, the handle 575b can extend outwardly from the table 575 to facilitate a user handling the handle 575b. In some embodiments, the handle 575b can extend at least a partial width of the table 575. In some embodiments, the handle 575b can extend substantially the entire width of the table 575. For example, in some embodiments, one end of the handle 575b can extend from the table 575 on one side, and another end of the handle 575b can extend from the table 575 on the opposite side. In some embodiments of the invention, the handle 575b can extend from the table 575 forming an aperture or cavity 579. In some embodiments, the aperture or cavity 579 can be used by a user to grip the handle 575b.

In some embodiments, the cart 520 can include wheel assemblies 527 or other conventional movement assemblies to enable the dental laser station 500 to be moved from one location to another location. In some embodiments of the invention, one or both of the transportation handles 575a, 575b can be used to maneuver the dental laser station 100 by moving on the wheel assemblies 527. For example, in some embodiments, the transportation handle 575a can be used to position the dental laser station 500. In some further embodiments, the transportation handle 575b can be used to position the dental laser station 100. In some embodiments, the wheel assemblies 527 can be locked to prevent unintended movement of the dental laser station 100. In some embodiments, the wheel assemblies 527 can be temporarily or permanently removed to enable the dental laser station 500 to remain stationary.

In some embodiments, the dental laser device 600 can include a display assembly 300 that can be pivoted or rotated by a user to enable a mounted or coupled display device to be raised or lowered for optimal viewing angle. Further, in some embodiments, the display assembly 300 can be pivoted or rotated so that the display device is positioned adjacent, proximate or flush with the main housing of the dental laser device 600. In some embodiments, the user can lower the assembly 300 to be positioned adjacent, proximate or flush with the main housing 601 of the dental laser device 600.

In some embodiments, the dental laser device 600 can include a transportation handle 605. In some embodiments, the transportation handle 605 can extend across the top end of the dental laser device 600. In some embodiments, the transportation handle 605 can extend across the top end of the device 600 from the front end to the back end of the main housing 601 of the device 600 as shown. In some embodiments, the handle 605 can extend across the top end of the device 600 from the front end to the back end of the main housing 601 forming an aperture 607. In some embodiments, the display assembly 300 can be coupled to a front portion of the main housing 601 of the transportation handle 605. In some embodiments, the display assembly 300 can be coupled to the front portion of a housing of the transportation handle using a pivot or other conventional mechanism to enable the display assembly 300 to rotate or pivot.

In some embodiments, the cart 520 can include a cabinet or drawer assembly 525 including a plurality of drawers 545 and cabinet space 540 for storage. Some embodiments include three drawers and a single cabinet. Some other embodiments can include additional or lesser numbers of drawers and/or partitions or other cabinets and spaces. In some embodiments, one or more drawers of the plurality of drawers 545 can be used to store supplies, tools, or accessories to assist in clinical practice using the dental laser station 500. For example, some embodiments include one or more laser handpiece tips, handpiece kit(s), glass, and/or a footswitch compartment.

It will be appreciated by those skilled in the art that while the invention has been described above in connection with particular embodiments and examples, the invention is not necessarily so limited, and that numerous other embodiments, examples, uses, modifications and departures from the embodiments, examples and uses are intended to be encompassed by the claims attached hereto.

1. A dental laser system comprising:
a mobile transportation cart including cabinet or drawer assembly and at least one support table;
an adjustment column coupled to the cabinet or drawer assembly;
at least one support table coupled to or mounted on the adjustment column, the at least one support table configured and arranged to be raised and lowered with respect cabinet or drawer assembly using the adjustment column; and
a dental laser assembly removably supported or mounted on the support table,
the dental laser assembly including at least one dental laser, at least one laser tool or handpiece coupled to the at least one dental laser, and at least one display assembly coupled to the at least on dental laser, wherein the distance between the dental laser assembly and the cabinet or drawer assembly is dependent on an adjusted position of the at least one support table.

2. The system of claim 1, wherein the cabinet or drawer assembly includes at least one drawer.

3. The system of claim 1, wherein the cabinet or drawer assembly includes at least one storage cabinet.

4. The system of claim 1, wherein the cabinet or drawer assembly includes at least one storage cabinet door.

5. The system of claim 1, wherein the mobile transportation cart comprises a set of wheels, the wheels enabling rolling movement of the cart by a user.

6. The system of claim 1, wherein the support table includes at least one transportation handle.

7. The system of claim 6, wherein the at least one transportation handle extends from the support table and forms an aperture.

8. The system of claim 6, wherein the at least one transportation handle extends from the support table generally perpendicular to the adjustment column.

9. The system of claim 6, wherein the at least one transportation handle extends from one side of the support table to the opposite side of the support table.

10. The system of claim 9, wherein the at least one transportation handle extends from one side of the dental laser assembly to the opposite side of the dental laser assembly.

11. The system of claim 1, wherein the at least one display assembly comprises a display of a computing device or controller.

12. The system of claim 11, wherein the computing device or controller comprises a mobile device that is attachable and detachable from the dental laser assembly.

13. The system of claim 1, wherein the mobile device comprises at least one of a laptop computer, tablet computer, or smart phone.

14. The system of claim 1, further comprising a laser tool mount configured to mount the laser tool or handpiece to the display assembly.

15. The system of claim 1, further comprising a display mount configured to enable mounting of the display assembly

16. The system of claim 15, wherein the display mount comprises at least one fixed or moveable lower grip.

17. The system of claim 1, wherein the display mount includes at least one retractable upper grip.

18. The system of claim 1, wherein the display assembly is configured to be tilted, pivoted or rotated on the dental laser assembly.

19. A dental laser system comprising:
a mobile transportation cart including a cabinet and a plurality of drawers;
a set of wheels enabling rolling movement of the cart by a user;
an adjustment column coupled to at least one height adjustable table, the at least one height adjustable table configured and arranged to be raised and lowered using the adjustment column; and
a dental laser assembly removably supported or mounted on the at least one height adjustable table,
the dental laser assembly including at least one dental laser, at least one laser tool or handpiece coupled to the at least one dental laser, and at least one display assembly mounted in a display mount of the at least on dental laser, wherein the distance between the dental laser assembly and the cabinet is dependent on an adjusted position of the at least one height adjustable table.

20. The system of claim 1, wherein the display assembly is configured to be pivoted, rotated or tilted, and the display mount includes at least one retractable or moveable grip.
